(19) United States
(12) Patent Application Publication
(10) Pub. No.: US 2013/0172834 A1
(43) Pub. Date: Jul. 4, 2013

(54) Fibrous Wound Filler Material for Negative Pressure Wound Therapy

(71) Applicant: David G. Heagle, Taunton, MA (US)

(72) Inventor: David G. Heagle, Taunton, MA (US)

(73) Assignee: TYCO HEALTHCARE GROUP LP, Mansfield, MA (US)

(21) Appl. No.: 13/551,963

(22) Filed: Jul. 18, 2012

Related U.S. Application Data
(63) Continuation of application No. 12/359,718, filed on Jan. 26, 2009, now abandoned.

(51) Int. Cl. A61F 13/00

(52) U.S. Cl. CPC A61F 13/00995; A61F 13/00068
USPC 604/319; 156/73.3

(57) ABSTRACT

An apparatus for promoting the healing of an exuding wound includes a wound cover for defining a reservoir over a wound in which a negative pressure may be maintained. The cover may form a substantially fluid-tight seal around the wound and permit fluid communication between the reservoir and a vacuum source suitable for providing an appropriate negative pressure to the reservoir to stimulate healing of the wound. A wound filler positioned between the wound and the wound cover includes a nonwoven material at least partially perforated by sonic welding.

### BACKGROUND

1. Technical Field

The present disclosure relates generally to wound dressings, and in particular to a fibrous wound filler material for improving wound exudates flow while reducing loose fiber contamination in a wound bed.

2. Background of Related Art

Wound dressings are generally placed over a wound to protect and promote healing of the wound. In the case of exuding wounds, such as pressure sores, ulcers and burns, it is customary to provide a dressing having an absorbent material or for absorbing at least a portion of the wound exudate as it is produced. Absorbing exudates promotes healing by removing potentially harmful bacteria from the wound bed, and also facilitates exudates removal from the wound bed via a vacuum system. Removal of excess exudates prevents damage to the surrounding skin that can be caused by an excessively moist environment.

The absorbent material temporarily stores the excess exudates until such time as they may be removed, by means of the vacuum system or as the dressing is periodically replaced with a new dressing. Because of the wide range of wound sizes that might be treated with a negative pressure wound therapy system, filler materials are commonly cut to custom fit the wound. In doing so, some absorbent materials such as cotton or foam tend to shed small fibers into the wound that may remain in the wound when the dressing is changed. Removing these stray fibers can be a labor intensive procedure that may be painful and further damage or cause trauma to the wound. Neglecting to remove these stray fibers may cause irritation, increase the risk of infection, and otherwise inhibit natural healing of the wound.

In negative pressure wound therapy (NPWT), the absorbent material may be positioned in a reservoir over the wound where a negative pressure may be maintained. The reservoir subjects the wound to a sub-atmospheric pressure to effectively draw wound fluid, including liquid exudates, from the wound without the continuous use of the vacuum pump. Hence, vacuum pressure may be applied once, or in varying intervals depending on the nature and severity of the wound. This technique has been found to promote blood flow to the area, stimulate the formation of granulation tissue, and encourage the migration of healthy tissue over the wound. An NPWT apparatus may also serve to draw exudates from the absorbent material out of the dressing without requiring that the entire dressing be changed. When an NPWT procedure is complete, the absorbent material must be removed and is thus subject to the difficulties that may be caused by stray fibers. Accordingly, an absorbent material suitable for use in wound dressings, including wound dressings adapted for use in advanced wound therapy procedures such as NPWT, would be helpful.

### SUMMARY

The present disclosure describes an apparatus for promoting the healing of an exuding wound. The apparatus includes a wound cover for defining a reservoir over a wound in which a negative pressure may be maintained. The cover may form a substantially fluid-tight seal around the wound and permit fluid communication between the reservoir and a vacuum source suitable for providing an appropriate negative pressure to the reservoir to stimulate healing of the wound. A wound filler positioned between the wound and the wound cover includes a nonwoven material at least partially perforated by sonic welding.

Methods of forming the perforated nonwoven wound filler are also described. In accordance with the present methods, a nonwoven material is passed through an ultrasonic welding device. The nonwoven material is sonically welded to fuse and to perforate the non-woven material. In embodiments, the ultrasonic welding device includes a patterned anvil for structuring the size and distribution of the perforations formed by the weld through the nonwoven wound filler.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is a cross sectional view of an NPWT apparatus incorporating a wound dressing formed in accordance with the present disclosure;

FIG. 2 is perspective view of a perforated nonwoven material which forms the wound filler of FIG. 1; and

FIG. 3 is a schematic view illustrating an exemplary process of forming the wound filler in accordance with the present disclosure.

### DETAILED DESCRIPTION OF THE EMBODIMENTS

The wound dressing of the present disclosure incorporates a perforated nonwoven wound filler suitable for improving exudates flow therethrough, while minimizing loose fiber formation. The nonwoven wound filler is sonically welded to bond and perforate the fibers of the nonwoven material to form apertures, free of protruding or loose fibers, through which exudates may flow. Because of the relatively fiber free surface provided by the sonic welding process, the nonwoven material will exhibit a substantially lower tendency to become attached to a healing wound bed. Further, the nonwoven fibers will have a substantially lower tendency to become separated from the wound filler and be inadvertently left in a wound when the wound dressing is changed.

While the specification refers to the use of a perforated nonwoven material as a wound filler for NPWT, the perforated nonwoven material may be used in a variety of wound care applications, such as a packing material for low exuding or shallow wounds.

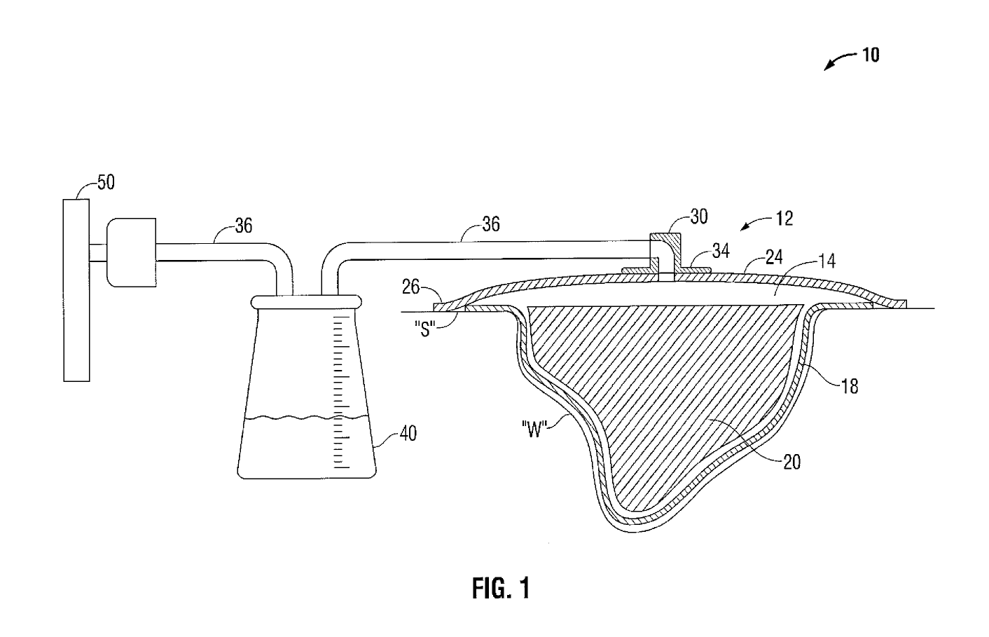

Referring initially to FIG. 1, an NPWT apparatus according to the present disclosure is depicted generally as 10 for use on a wound “w” surrounded by healthy skin “s.” The NPWT apparatus 10 includes a wound dressing 12 positioned relative to the wound “w” to define a reservoir 14 in which a negative pressure appropriate to stimulate healing may be maintained.

Wound dressing 12 may include a contact layer 18 positioned in direct contact with the bed of wound “w” and may be formed from perforated film material. An appropriate perforated material permits the negative pressure applied to the reservoir to penetrate into the wound “w,” and also permits exudates to be drawn through the contact layer 18. A non-adherent material may be selected such that contact layer 18 does not tend to cling to the wound “w” or surrounding tissue when it is removed. One exemplary material that may be used as a contact layer 18 is sold under the trademark XEROFLO® by Tyco Healthcare Group LP (d/b/a Covidien), or the commercially available CURITY non-adherent dressing offered by Tyco Healthcare Group LP (d/b/a Covidien). This dressing is an open mesh knitted fabric material made from a cellulose acetate and impregnated with a petrolatum emulsion.

Wound filler 100 is positioned in the wound “w”, over the optional contact layer 18, and is intended to allow wound dressing 12 to transfer wound exudates. Wound filler 100 is conformable such that it may assume the shape of any wound “w” and may be packed up to the level of healthy skin “s.” As discussed in greater detail below, the wound filler 100 may be formed from a perforated nonwoven material.

Wound dressing 12 also includes a cover layer 24 in the form of a flexible membrane. Cover layer 24 may be positioned over the wound “w” such that a biocompatible adhesive at the periphery 26 of the cover layer 24 forms a substantially fluid-tight seal with the surrounding skin “s.” Thus, cover layer 24 may act as both a microbial barrier to prevent contaminants from entering the wound “w,” and also a fluid barrier maintaining the integrity of vacuum reservoir 14. Cover layer 24 is preferably formed from a moisture vapor permeable membrane to promote the exchange of oxygen and moisture between the wound “w” and the atmosphere. A membrane that provides a sufficient moisture vapor transmission rate (MVTR) is a transparent membrane sold under the trade name POLYSKIN®II by Tyco Healthcare Group LP (d/b/a Covidien). A transparent membrane permits an assessment of wound conditions to be made without requiring removal of the cover layer 24. Alternatively, cover layer 24 may comprise an impermeable membrane 24. As a further alternative, cover layer 24 may be substantially rigid.

A vacuum port 30 having a flange 34 may also be included in wound dressing 12 to facilitate connection of the wound dressing 12 to fluid conduit 36. Fluid conduit 36 defines a fluid flow path leading through the apparatus 10. The vacuum port 30 may be configured as a rigid or flexible, low-profile component, and may be adapted to receive a vacuum tube 36 in a releasable and fluid-tight manner. An adhesive on the underside of flange 34 may provide a mechanism for affixing the vacuum port 30 to the dressing 12, or alternatively flange 34 may be positioned within reservoir 14 (not shown) such that an adhesive on an upper side of the flange 34 affixes the vacuum port 30. However it is affixed to the dressing, a hollow interior of the vacuum port 30 provides fluid communication between the fluid conduit 36 and the reservoir 14. Vacuum port 30 may be provided as a pre-affixed component of dressing 12, as a component of fluid conduit 36 or entirely independently. Alternatively, vacuum port 30 may be eliminated from dressing 12 if other provisions are made for providing fluid communication with the fluid conduit 36.

Fluid conduit 36 extends from the vacuum port 30 to provide fluid communication between the reservoir 14 and collection canister 40. Any suitable conduit may be used for fluid conduit 36 including those fabricated from flexible elastomeric or polymeric materials. Fluid conduit 36 may connect to the vacuum port 30, the canister 40, or other apparatus components by conventional air tight means such as friction fit, bayonet coupling, or barbed connectors. The conduit connections may be made permanent, or alternatively a quick-disconnect or other releasable means may be used to provide some adjustment flexibility to the apparatus 10.

Collection canister 40 may comprise any container suitable for containing wound fluids. For example, a rigid bottle may be used as shown or alternatively a flexible polymeric pouch may be appropriate. Collection canister 40 may contain an absorbent material to consolidate or contain the wound drainage or debris. For example, super absorbent polymers (SAP), silica gel, sodium polyacrylate, potassium polyacrylamide or related compounds may be provided within canister 40. At least a portion of canister 40 may be transparent to assist in evaluating the color, quality or quantity of wound exudates. A transparent canister may thus assist in determining the remaining capacity of the canister or when the canister should be replaced.

Leading from collection canister 40 is another section of fluid conduit 36 providing fluid communication with vacuum source 50. Vacuum source 50 generates or otherwise provides a negative pressure to the NPWT apparatus 10. Vacuum source 50 may comprise a peristaltic pump, a diaphragmatic pump, or other mechanism that is biocompatible and draws fluids, e.g. atmospheric gases and wound exudates, from the reservoir 14 appropriate to stimulate healing of the wound “w.” Preferably, the vacuum source 40 is adapted to produce a sub-atmospheric pressure in the reservoir 14 ranging between about 20 mmHg and about 500 mmHg, more preferably, about 75 mmHg to about 125 mmHg, and, in embodiments, about 40 mmHg to about 80 mmHg.

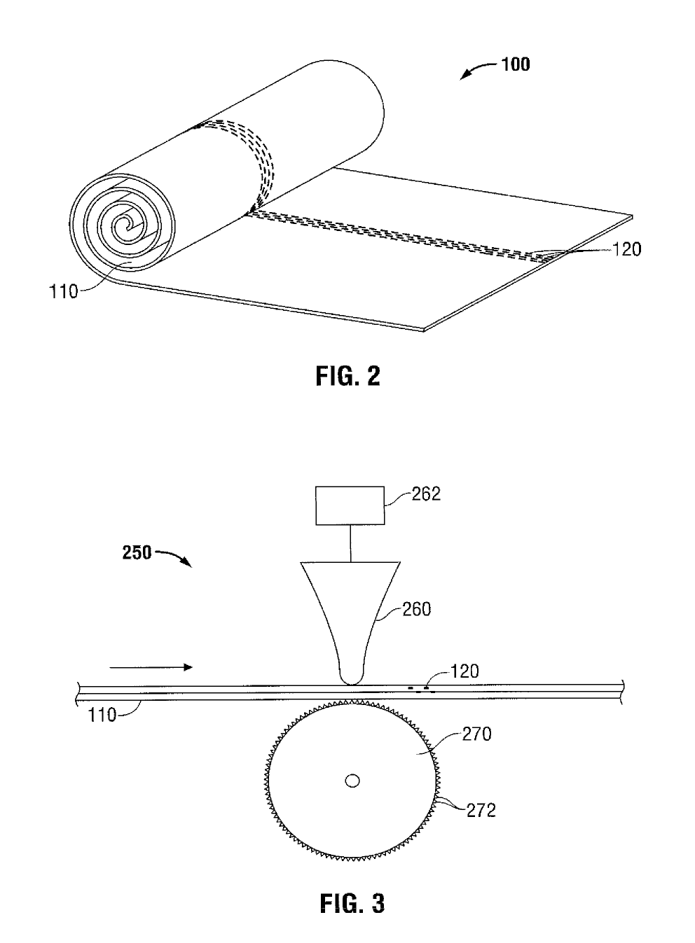

Referring now to FIG. 2, a wound filler 100 may be formed of a nonwoven material 110 including perforations or apertures 120. The nonwoven material 110 may be adapted to absorb wound fluid and exudates, or may be adapted to convey or wick fluids or exudates from the wound bed for removal by vacuum source 40. Nonwoven material 110 may be a continuous filament fiber or a mass of fibers of a natural, synthetic, or composite material, randomly or systematically arranged and/or coupled together to form a batting having a desirable loft. The fiber(s) may be formed into a sheet or web, and then bound mechanically by matting, pressing, needle punching, or otherwise interlocking the fiber(s); chemically by use of an adhesive; or thermally by applying a binder, such as a powder, paste, or melt, and melting the binder onto the sheet or web.

Wound filler 100 may be resilient and compressible so that it can easily conform and assume the shape of any wound “w”, such as an irregular-shaped wound bed. Wound filler 100 may be any commercially available nonwoven material. The nonwoven material may be comprised of absorbent and/or non-absorbent materials and may include, for example, polyolefins such as polypropylene and polyethylene; polyesters such as polyethylene terephthalate; polyamides such as nylon; siloxanes such as silicone; and fluoropolymers such as polyterafluoroethylene. Exemplary materials that may be used as a nonwoven material 110 are continuous filaments of spun bound and needle punched polyester, such as Type 021/200, by Johns Manville Engineered Products Division of Johns Manville (Spartanburg, S.C.).

The wound filler 100 may be formed in any shape and size. For example, the wound filler 100 may be a pre-formed shape, such as square or circle sponges, of various sizes. The wound filler 100, as illustrated in FIG. 2, may be stored or maintained as a roll. In embodiments, the wound filler 100 may be assembled into 2 inch rolls, but the wound filler 100 may be formed into rolls of any width, length, and size.

The nonwoven material 110 may be sonically welded to prevent fibers from separating therefrom and/or to provide perforations therethrough. The perforations or apertures 120 permit the negative pressure applied to the reservoir to penetrate into the wound “w,” and also permits exudates to be drawn through the wound filler 100. Sonic welding involves the use of high frequency sound waves to melt material and cause the material to flow together and mechanically bond. Typically, the source of the sound waves is a sound-generating metal tuning device known as a horn that converts a high-frequency electric signal into sound, although any suitable source of sonic sound may be used. Commercially available sonic welding machines may be utilized for welding and perforating nonwoven material 110.

Turning now to FIG. 3, a schematic is shown for ultrasonic welding and perforation of a nonwoven material to form the wound filler of the present disclosure. Ultrasonic welding device 250 includes horn 260 and anvil 270. Anvil 270 is shaped as a cylindrical drum having raised projections 272. Projections 272 provide small contact surfaces so that the energy delivered by ultrasonic welding device 250 is concentrated over a small area. The projections 272 of anvil 270 may be any shape, such as, for example, rectangular, triangular, circular, oval, and other polygons and irregular shapes and combinations thereof. The anvil 270 may also include a pattern of projections 272 for structuring the size and distribution of the perforations within nonwoven material 110. For example, the anvil may be patterned so that perforations are partially formed in the nonwoven material, such as only on one side, or the perforations may extend completely throughout the nonwoven material in an even or random distribution depending upon the anvil pattern utilized. In embodiments, the perforations are about 0.01 inches to about 0.25 inches in length, in some embodiments, about 0.094 inches in length.

The nonwoven material 110 is passed over anvil 270 and mechanically worked by moving horn 260 up and down via driving means 262 into portions of nonwoven material 110 lying on projections 272 with a frequency that lies within the ultrasonic range. Heat is generated in the worked areas of the material causing the material to melt and fuse together. The heat generated will perforate the nonwoven material 110 and form apertures 120 while fusing the fibers lying in the periphery of the aperture 120 so that no stray fibers are produced.

In the alternative, the amount of heat generated may be lower to affect only melting and heating of the fibers of nonwoven material 110, such that the nonwoven material must be perforated using a separate tool, such as a punch, at the welded sites.

Any combination of steps as described above may be utilized to bind the fibers of the nonwoven material and provide apertures therethrough. Larger areas of the nonwoven material may be sonically welded by providing additional horns or larger horns to the welding device or by using a flat anvil.

The wound filler of the present disclosure may further be used for delivery of a bioactive agent. The bioactive agent may be any substance or mixture of substances that have clinical use. Consequently, bioactive agents may or may not have pharmacological activity per se, e.g., a dye. Alternatively a bioactive agent could be any agent that provides a therapeutic or prophylactic effect, a compound that affects or participates in tissue growth, cell growth, cell differentiation, a compound that may be able to invoke a biological action such as an immune response, or could play any other role in one or more biological processes. It is envisioned that the bioactive agent may be applied to the wound filler in any suitable form of matter, e.g., films, powders, liquids, gels and the like.

Examples of classes of bioactive agents which may be utilized in accordance with the present disclosure include anti-adhesives, antimicrobials, antibacterials, antiobiotics, anti-virals, anti-fungals, anti-septics, anti-inflammatories, and anesthetics. It is also intended that combinations of bioactive agents may be used. For example, an anti-adhesive, to prevent adhesions from forming between the wound filler and the surrounding tissue, may be utilized with an antimicrobial, such as polyhexamethylene biguanide, to reduce the bio burden in the wound bed.

While the disclosure has been illustrated and described, it is not intended to be limited to the details shown, since various modifications and substitutions can be made without departing in any way from the spirit of the present disclosure. As such, further modifications and equivalents of the disclosure can occur to persons skilled in the art, and all such modifications and equivalents are intended to be within the spirit and scope of the disclosure as defined by the following claims.

What is claimed is:

1. An apparatus to promote the healing of an exuding wound comprising:
a wound cover for defining a reservoir over a wound in which a negative pressure may be maintained by forming a substantially fluid-tight seal around the wound;
a vacuum source in fluid communication with the reservoir, the vacuum source suitable for providing an appropriate negative pressure to the reservoir to stimulate healing of the wound; and
a wound filler positioned between the wound and the wound cover, the wound filler comprising a nonwoven material at least partially perforated by sonic welding.

2. The apparatus according to claim 1, wherein the nonwoven material comprises a continuous filament.

3. The apparatus according to claim 1, wherein the nonwoven material is made of polyester.

4. A method of forming a perforated nonwoven material wound filler comprising the steps of:
providing a nonwoven material; and
sonically welding the nonwoven material thereby fusing and perforating the nonwoven material.

5. The method of claim 4, wherein the step of sonically welding the nonwoven material includes the step of:
using a patterned anvil including projections for forming the perforations in the nonwoven material.
